(19) United States
(12) Patent Application Publication
(10) Pub. No.: US 2004/0249294 A1
(43) Pub. Date: Dec. 9, 2004

(54) Method for Identifying Individuals

(75) Inventor: Manoj THULASIDAS, Singapore (SG)

Correspondence Address: SUGHRUE MION, PLLC, 2100 PENNSYLVANIA AVENUE, N.W., SUITE 800, WASHINGTON, DC, 20037, US

(73) Assignee: AGENCY FOR SCIENCE, TECHNOLOGY AND RESEARCH, Singapore (SG)

(21) Appl. No.: 10/250,125

(22) Filed: Jun. 5, 2003

(51) Int. Cl. A61B 5/0402

(52) U.S. Cl. 600/509

(57) ABSTRACT

A method and system for identifying a subject comprises obtaining a digitised recording of an electrocardiogram measurement of the subject to be identified, the digitised recording being a cyclic waveform having a peak amplitude. The digitised recording is normalised to reduce variations due to physiological effects, and the normalised recording is processed to determine a feature vector in the frequency domain. The distance between the determined feature vector and a predetermined feature vector is measured to identify the subject.

### BACKGROUND OF INVENTION

The present invention relates to methods for identifying human individuals. The term “identifying individuals” is used particularly to include detecting or confirming the identity of a given individual.

A variety of methods are known to identify human individuals, ranging from fingerprint identification to voice identification. There is a risk of fraudulent reproduction in each method. Many such methods also suffer from the drawback that the results are unreliable (e.g. because at different times humans have different vocal characteristics) or require relatively sophisticated measurement devices (e.g. a finger print sensor).

One conventional technique [3] proposes the measurement of a subject's heart beat profile and the extraction of characteristics of the heart beat profile which are then compared to a database of heart beat profiles from a plurality of individuals to identify whether the individual is one of the plurality of individuals and, if so, the identity of the subject.

The heart beat profiles used in the technique described in [3] are electrocardiograms (conventionally abbreviated as ECG or EKG). In this technique, extracting facilities are contained in the ECG machine for extracting a number of selected features of a subject's ECG readings. The selected features are processed by soft independent modeling of class analogy (SIMCA ) which applies automatic clustering algorithms to the data to build a principal component analysis model enabling the identification of sample subjects.

One disadvantage of the system described in [3] is that it requires a complicated ECG machine fitted with the feature selection equipment and supervised operation and training to operate the machine.

### SUMMARY OF INVENTION

The present invention aims to provide a new and useful method of identifying individuals based on the concept that the ECG of a subject shows a characteristic constancy which is sufficient for the individual to be identified, although it also includes a variability due to a number of factors (both known and unknown). Preferably, the present invention aims to provide a method of identifying individuals by processing the output from an ECG machine such that variations are normalised, or modeled, to accentuate the constant characteristics in the signal.

In general terms, the present invention in a first aspect proposes a method for identifying a subject comprising the steps of:

(a) obtaining a digitised recording of an electrocardiogram measurement of the subject to be identified, said digitised recording being a cyclic waveform having a peak amplitude;

(b) normalising the digitised recording for reducing variations due to physiological effects;

(c) processing the normalised recording to determine a feature vector in the frequency domain; and

(d) measuring the distance between the determined feature vector and a predetermined feature vector to identify the subject.

Preferably, the step of normalising comprises normalising the peak amplitude of the digitised recording.

In a preferred embodiment, if the digitised waveform has a DC component, the step of normalising comprising removing the DC component.

In a preferred embodiment, the step of normalising comprises taking an average of the peak amplitude of a number consecutive cycles to reduce statistical errors, and, preferably, prior to taking the average of the peak amplitude, aligning the peaks of the cyclic waveforms.

Preferably, the step of normalising comprises resampling the digitised recording, for example, using FFT resampling techniques.

Preferably, the step of normalising comprises obtaining a quadrature distance for each cycle against a running average of the cycles and rejecting a cycle when the distance is more than a predetermined distance until a predetermined number of cycles has been averaged.

In a preferred embodiment the step of processing comprises processing the normalised recording to determine a frequency response and may comprise obtaining the quadratic distance between the feature vector and a predetermined vector.

Preferably, the step of measuring includes obtaining the quadratic distance between the feature vector and a predetermined vector, applying a correction for standard deviations in both vectors to obtain an error-compensated distance and comparing the error-compensated distance with a predetermined value to identify the subject.

According to a further aspect of the invention there is provided a system for identifying a subject using a digitised recording of an electrocardiogram measurement of the subject to be identified, said digitised recording being a cyclic waveform having a peak amplitude the system comprising:

(a) a normalising device for normalising the digitised recording;

(b) a processor for processing the normalised recording to determine a feature vector in the frequency domain, the processors being arranged to measure the distance between the determined feature vector and a predetermined feature vector to identify the subject.

Preferably, the system for identifying a subject using a digitised recording of an electrocardiogram measurement of the subject to be identified, is arranged to carry out the method defined above.

### BRIEF DESCRIPTION OF DRAWINGS

Preferred features of the invention will now be described, for the sake of illustration only, with reference to the following figures in which:

FIG. 1 is a graph showing the variation of voltage output over one cycle of an ECG and the different diagnostic feature points;

FIGS. 2a, 2b, 2c and 2d are graphs showing the variation of voltage output over a number of cycles of an ECG at different stages of the analysis of the voltage output;

FIG. 3 is a graph showing a distribution of metric distances (Q_{d}) between an ECG cycle and the average ECG cycle used to compute the metric;

FIG. 4 shows the distribution of normalised errors with a Gaussian distribution superimposed thereon;

FIG. 5 is a graph showing the frequency components of the metric obtained from a typical ECG cycle;

FIG. 6 is a graph showing, for two conditions, a distribution of metric distances (Q_{d}) against the probability that the metrics are taken from the same subject, one condition being where the measurements are taken from the same subject and the other condition being where the measurements are taken from different subjects;

FIG. 7a is a graph showing, for two conditions, a distribution of metric distances (Q_{d}) against the probability that the metrics are taken from the same subject, one condition being where the measurements are taken from the same subject and the other condition being where the measurements are taken from different subjects with samples taken at intervals of more than one hour;

FIG. 7b is a graph showing a distribution of distances between metrics allowing for statistical errors ( [symbol] ²) against the probability that the metrics are taken from the same subject, one condition being where the measurements are taken from the same subject and the other condition being where the measurements are taken from different subjects with samples taken at intervals of more than one hour; and

FIG. 8 is a graph showing a distribution of distances between metrics allowing for statistical errors ( [symbol] ²) against the probability that the metrics are taken from the same subject, one condition being where the measurements are taken from the same subject and the other condition being where the measurements are taken from different subjects.

### DETAILED DESCRIPTION

There have been a number of advances in ECG (electrocardiogram) measurement techniques recently which enhance both the accuracy and convenience of the data collection [1]. Such techniques have made it possible to identify, for example, that an individual's ECG depends upon the orientation of the individual's heart [2].

Such dependence suggests that an identifying signature based on ECG can be robust against fraudulent reproduction. The previous work in this direction [3] used supervised clustering techniques on time domain parameters and reported encouraging results. We employ robust frequency domain techniques after eliminating or minimizing known sources of variabilities.

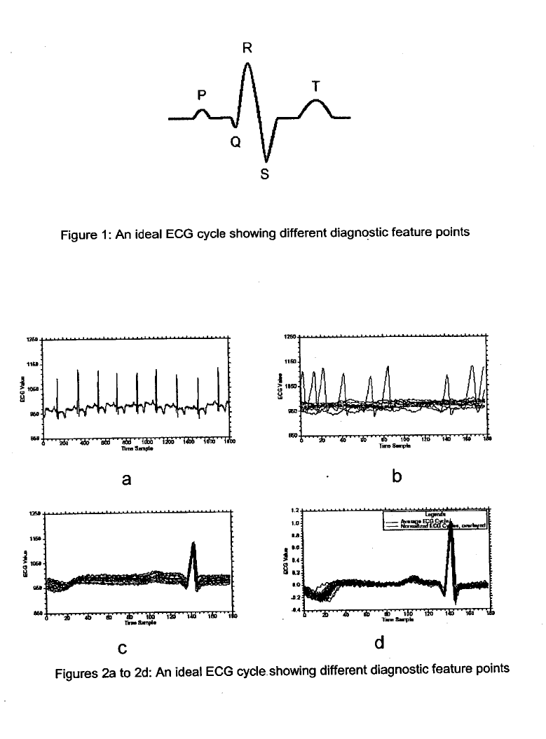

An Electro-Cardiogram is a representation of the heart's electrical activities. Typically, an ECG measures and records different electrical potentials on the surface of the human body. These potentials arise from the electrical activities of the heart. An ECG cycle may roughly be divided into the phases of depolarization and repolarization of the muscle fibers making up the heart. The depolarization phases correspond to the P-wave (atrial depolarization) and QRSwave (ventricles depolarization). The repolarization phases make up the T-wave and U-wave (ventricular repolarization). The different peaks of the ECG-complex are shown in FIG. 1.

An ECG is typically measured by placing ten electrodes on selected spots on the human body surface. The most common ECG measurement makes use of 10 electrodes placed on the body. Out of these ten electrodes, six electrodes are placed on the chest, and four electrodes are placed on the extremities. A comprehensive discussion of Electro-Cardiogram and measurement principles can be found in [4]. The electrical potential differences in 12 different directions out of the ten electrodes are measured.

These 12 different electrical views of the activity in the heart are normally referred to as leads. The 12 leads are made up of three bipolar and nine monopolar leads. The three bipolar leads are the electrical potentials between the right and left arm (lead I), the right arm and left foot (lead II), and between the left arm and left foot (lead III).

For the monopolar leads, four different artificial reference points are constructed. These reference points are the average of the signals seen at two or more electrodes. Using these reference points, the potentials appearing on the left arm (aVL), the right arm (aVR), the left foot (aVF), and on the six chest electrodes (V1-V6) are measured. The right foot is normally used for grounding purposes only.

It is true that there is substantial variability among individuals, as demonstrated by several studies [5, 6, 7]. This inter-individual variability is a problem from a medical diagnostic point of view. Furthermore, there is an intra-individual variability. For example, one study [8] has reported the effects of exercises on the shape of the ECG cycle (i.e. normalized slopes) in addition to the more palpable variations in overall rate and amplitude.

The method proposed by the present invention does not model the variations in slope of the ECG cycle, but concentrates on the qualitative constancy in the shape, and formalizes ways of reducing the intra-individual variability to a point where it is not significant.

An ECG depends on physical conditions such as exertion, medical condition such as fever and emotional states such as anger or fear. In addition to such intrinsic variations, the measurement techniques introduce another set of uncertainties. Noise pickup, electrode position and conductive differences all contribute to the intra-individual variability. There are also statistical effects to worry about.

The statistical errors in the ECG signal are minimised by taking an average of a number of consecutive ECG cycles. Note that a simple minded averaging based on the average time period does not work in the case of bio-electric signals because of the tiny uncertainties in the time period (see FIG. 2(b)). It is necessary to line up the different ECG cycles at some prominent feature point. In a preferred embodiment of the invention the QRS complex (see FIG. 1) is used to line up different cycles. The algorithm described in [9] is used to search for the QRS complex in a cycle.

The systematic variations (slow drifts, calibration issues) are handled by normalizing the amplitude. The R peak is normalised to have a value of one and ensure that the average DC component is zero. It is important to have zero DC value as a Fourier analysis is used in the frequency domain and non-zero DC value induces artificial differences in any distance measured between spectra.

The variations due to (non-medical) physiological reasons typically show up as changes in the fundamental frequency, i.e. the heart beats faster or slower. In order to normalise this systematic variability, the averaged cycle is “stretched” or “compressed” to a constant time period using resampling techniques. Resampling is trivial in the frequency domain. Sampling up implies padding the frequency spectrum with zeros, and sampling down is the same as band-limiting.

In order to reduce known variabilities further, ECG cycles are validated before averaging, i.e. ensuring that they are not very different from each other. This is done by studying the quadrature distance between each cycle used in computing the average ECG cycle and the running average.

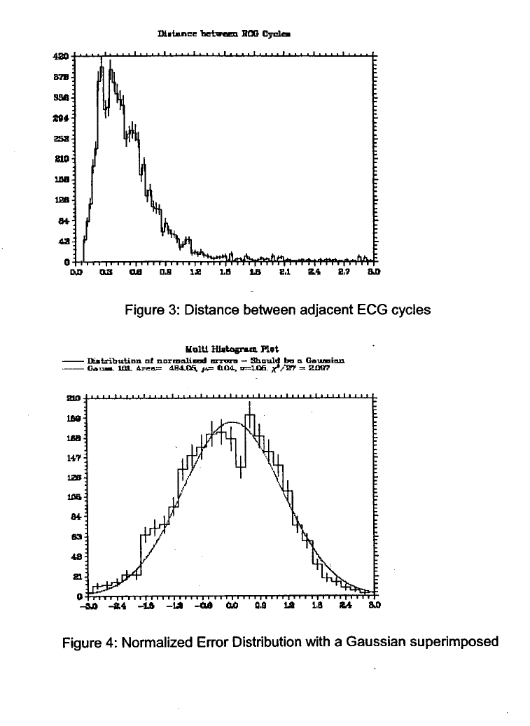

FIG. 3 shows the distribution of the quadrature distance between an ECG cycle and the average ECG cycle used to compute the metric. Based on this distribution, it is preferred that the distance be less than 1 before averaging is performed. This selection criterion removes about 10% of the cycles and improves the statistical quality of the metric.

The data sample used in this work was proposed by Physionet [10] and the tools available therefrom for accessing the data were used in preferred embodiments of the present invention. The records were visually inspected and it was subjectively decided which records to use. The decision was based on the shape of the ECG being close to an ideal ECG (FIG. 1). A qualitatively reasonable definition of the QRS complex is required for the algorithm used in preferred embodiments of the present invention to work. For the general studies, the QT Database was used, which contains a total of 105 fifteen-minute excerpts of two channel ECGs. A detailed description of this database is available in [11]. Out of these, 84 samples were chosen for the following studies. From each ECG signal, multiple sections of 10 cycles were studied, each at about 40 second intervals.

For robustness studies, the MIT-BIH Normal Sinus Rhythm Database was used. This database includes 18 long-term ECG recordings of subjects referred to the Arrhythmia Laboratory at Boston's Beth Israel Hospital (now the Beth Israel Deaconess Medical Center). Subjects included in this database were found to have had no significant arrhythmias; they include 5 men, aged 26 to 45, and 13 women, aged 20 to 50. 11 of these 18 data sets were used for the present robustness studies. Multiple sections of about 10 cycles at more than 1 hour interval (100 times longer than the general studies) were studied and the metrics between same and different persons were compared.

An ideal cardiac cycle is shown in FIG. 1. A measured ECG cycle is quite a bit more noisy. The shape and constancy of a measured cycle (e.g. FIG. 2(a)) depend on measurement effects, electrode placement, noise, etc. The different steps in the analysis are aimed at getting to the ideal picture starting from a measurement.

After reading the data, an attempt is made to minimize statistical effects by averaging over a number of cycles (10 in the current version). As shown in FIG. 2(b), a straightforward wrapping around of the cycles using the average time period is disastrous. The QRS complex of each ECG cycle is lined up before taking an average. This results in a waveform shown in FIG. 2(c). Note that even in this, there is some systematic variation between cycles, as evidenced by the thickness of the overlaid curves. This is minimized by normalising each cycle so that the R peak is at 1 and the average DC is zero. This is shown in FIG. 2(d). Also shown on FIG. 2(d) is the averaged waveform.

Any selection on a statistical ensemble can skew the error distribution and bias the conclusions. Hence, it is necessary to verify that the error distribution is normal after the selection in validating the ECG cycles.

FIG. 4 shows the distribution of normalized deviations from the mean for each point in the ECG cycles. If x is the measurement, Î¼ the mean and Ï· the estimated standard deviation, then the distribution plotted is that of Î′.

$$δ = \frac{x - μ}{σ} \qquad ( 1 )$$

If the Ï· is well estimated and the errors are unbiased, then the distribution is supposed to be a normal Gaussian. Superimposed on the distribution in FIG. 4 is a free fit to a Gaussian. The fit values correspond well to a normal distribution, verifying that the errors are not skewed in anyway.

Once the average typical ECG cycle is obtained with normalised amplitude, it is necessary to consider the variations in the time axis. Such variations are likely to come from physiological causes. In preferred embodiments of the present invention, time period variations are handled by stretching or compressing the wave to a standard time period using FFT resampling techniques.

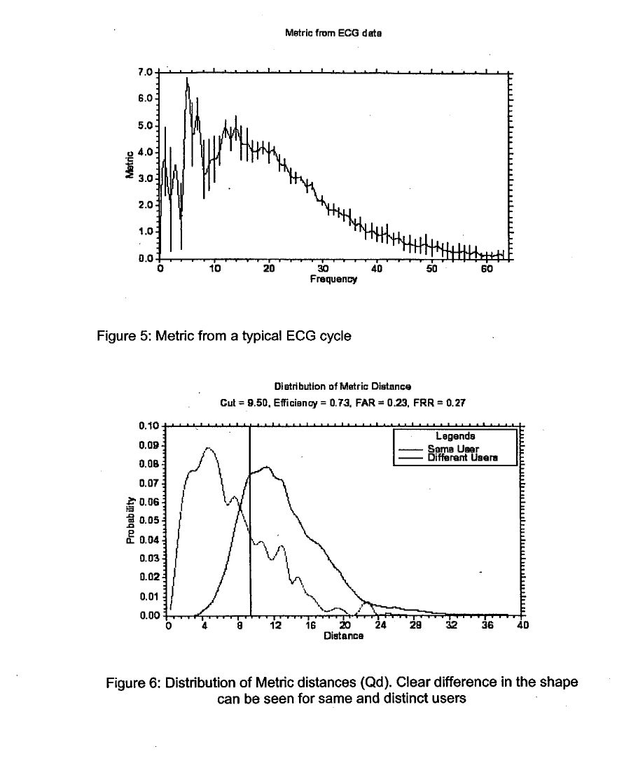

Then a metric is defined as the first 64 components of the frequency spectrum. Note that the spectrum is well behaved and has zero value at 0 Hz since we normalised the ECG cycle. Also, note that the spectrum is independent of time shifts in the averaged cycle, i.e. the QRS complex can be anywhere within the period without affecting the spectrum. Despite this property, in a preferred embodiment of the present invention, the average cycle was rotated so that the QRS complex always falls at a certain fixed position. This is done so to enable the extraction of more information from the phase spectrum later. FIG. 5 shows a typical metric.

In order to estimate the statistical significance of any measure of distance between two metrics, it is essential to understand the errors associated with the metric. The standard deviation of the 10 cycles of ECG used to compute the metric is a measure of the statistical error associated with the ECG measurement. It can be shown that the errors on the metric are completely determined by the errors on the typical, averaged ECG cycle and the phase response of the FFT. The following describes how these errors can be propagated to the metric. Once the errors are understood, it is possible to construct an [symbol] ² and use it as a measure of distance between two metrics. Consider a signal in the time domain defined by n time samples and the corresponding errors. Using a vector notation, it can be represented as X Â±X. (X is an n dimensional vector). The FFT of X is to be referred to as Y. The Fast Fourier Transform (FFT) of X is defined as:

Y=FFT(X)⁽²⁾

In terms of the components,

$${y}_{k} ≡ {\sum }_{j = 0}^{n - 1} {x}_{j} {ⅇ}^{\frac{ⅈ2π k j}{n}} \qquad ( 3 )$$

The errors on Y are Î·Y. Taking the differential of Equation (3), these errors may be computed as

$$Δ Y = {δ}_{{y}_{k}} = δ {\sum }_{j = 0}^{n - 1} {x}_{j} {ⅇ}^{\frac{ⅈ 2 π kj}{n}} = {\sum }_{j = 0}^{n - 1} δ {x}_{j} {ⅇ}^{\frac{ⅈ 2 π k j}{n}} = FFT ( Δ X ) \qquad ( 4 )$$

The metric is the frequency response F and the errors on the components fk are of interest. F can be expressed in n dimensional vector notation which translates to component notation as

Y≡Fe^{iφ}

which translates to component notation

yₖ= [symbol] (yₖ)+iℑ(yₖ)=fₖ cos φₖ+ifₖ sin φₖ (5)

Equation (5) defines Î¦ₖ by equating real and imaginary parts. Also,

F≡|Y|=\sqrt{ [symbol](Y)²+ℑ(Y)²} (6)

Equation (6) can be rewritten in terms of the components as

∫²ₖ− [symbol] y_{A})²−ℑ(y_{A})² (7)

The errors on yₖ may be propagated to those in fₖ by taking the differentials of Equation (7)

|fₖδfₖ= [symbol] (yₖ)δ [symbol] (yₖ)|+|ℑ(yₖ)δℑ(yₖ)|= [symbol] (yₖ) [symbol] (δyₖ)|+|ℑ(yₖ)ℑ(δyₖ)|(

Using [symbol] (yₖ)=fₖ cos φ and ℑ(yₖ)=fₖ sin φₖ from Equation (5)

|δfₖ|=| [symbol] (δyₖ)cos φₖ|+|ℑ(δy^{k})sin φₖ|

Substituting for Î′yₖ from Equation (4), and going back to the vector notation:

ΔF= [symbol] (ΔY)cos Φ+ℑ(ΔY)sin Φ= [symbol] (FFTΔX)cos Φ+ℑ(FFTΔX)sin Φ

Thus, the errors on the metric (F) can be completely calculated from the errors on the typical, averaged ECG cycle (X) and the phase response of the FFT. The standard deviation of the 10 cycles of ECG used to compute the metric is a measure of the statistical error Î·X. This is propagated and Î·F computed as described above.

Since the frequency components define an orthogonal basis, the metric may be thought of as a vector in a 64 dimensional space. Then a measure of distance between two metrics may be defined as the quadrature distance in the 64 dimensional space. The Quadratic Distance (Qd) between two metrics (_{\vec{M}1} and _{\vec{M}2} whose components are M1j and M2j) is defined as:

$${Q}_{d} ≡ \sqrt{{\sum }_{j = 1}^{64} {( {M}_{1 j} - {M}_{2 j} )}^{2}}$$

For ECGs from the same person at different times, the distribution of this metric distance should peak around zero. For different people, the peak should be at a positive value. FIG. 6 shows these two distributions, which confirm expectations. As the statistical errors to the metric have been propagated, a more accurate measure of the significance of the distance may be defined as the [symbol] ² difference between the metrics. The [symbol] ² is defined as

$${χ}^{2} ≡ {\sum }_{j = 1}^{64} \frac{{( {M}_{1 j} - {M}_{2 j} )}^{2}}{{σ}_{1 j}^{2} + {σ}_{2 j}^{2}} \qquad ( 10 )$$

where, in addition to the symbols used in Equation (1), the errors on the components of the two metrics Ï·₁ⱼ and Ï·₂ⱼ are included

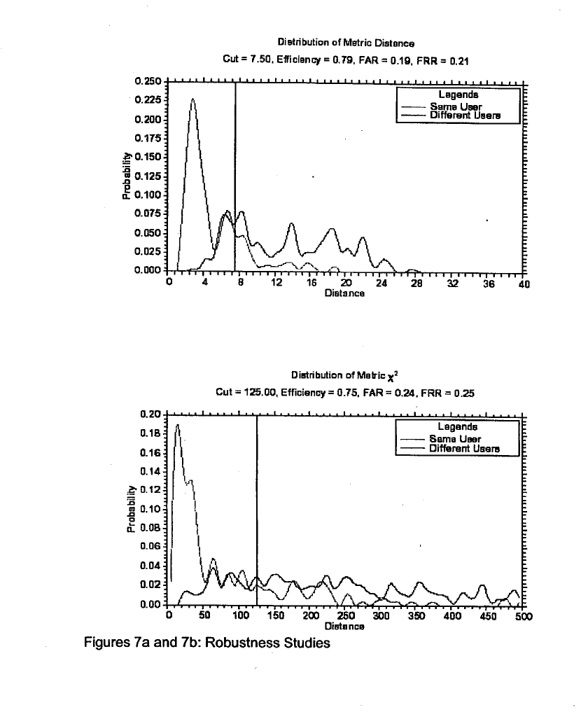

For two ECGs from the same person at different times, it is anticipated that the distribution of this metric distance will peak around zero. For different people, the peak should be at a positive value. FIGS. 7a and 7b show these two distributions, which confirm expectations.

The analysis has the following steps:

1. The ECG data is read.

2. The time period is dynamically recomputed, and the beginning of each ECG cycle is identified by matching the qrs complex.

3. The amplitude in each cycle is normalised so that the R peak is 1.0 and the DC value is zero.

4. The data is wrapped at the dynamically recomputed periods so that the R peaks overlie each other.

5. The average ECG (and the statistical error on it) is found by summing up the overlying ECG cycles.

6. The time period is normalised to a constant.

7. The frequency spectrum (and the error) of the average, normalised ECG cycle is computed.

8. The frequency response of the normalised average cycle is compared to study the inter- and intra-individual variability.

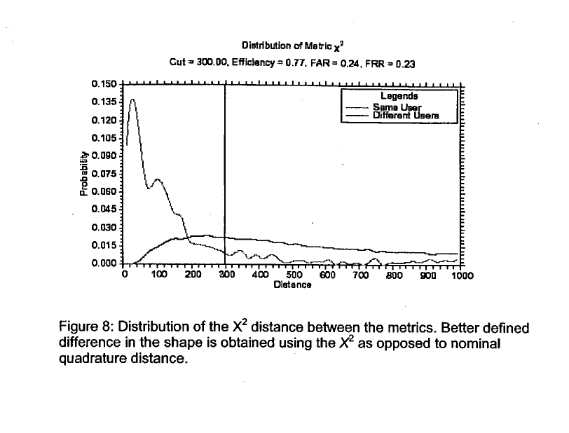

FIGS. 6 and 8 show the results of the Applicant's studies as histogram distributions of the distance measures (Quadrature Distance in FIGS. 6 and [symbol] ² distance in FIGS. 8.) On the X axis is plotted the distance value and on the Y axis is plotted the number of times such a distance value is obtained. Since the areas under each histogram is normalized to unity, these distributions represent the probability density functions of Qd and [symbol] ². In each figure, the Qd and [symbol] ² from same person's ECG taken at different times and different people's ECG have been super-imposed. With an arbitrary selection criterion (the “cut” value chosen to coincide where the two curves intersect, for example) on Qd or [symbol] ², it is possible to compute the Efficiency, False Acceptance Rate and False Rejection Rate as defined below.

Efficiency describes how often the method embodying the present invention succeeds in identifying the right person using ECG metric. It is defined as the fraction of the right combinations accepted. It corresponds to the area of the right combination curve below the “cut” value.

False Acceptance Rate FAR describes how often the method embodying the present invention falsely identifies a wrong person using ECG metric. It is the fraction of the wrong combinations accepted. It corresponds to the area of the wrong combination curve below the “cut” value.

False Rejection Rate FRR is a measure of the frequency of the right person being rejected. It corresponds to the area of the right combination curve above the “cut” value.

Equal Error Rate EER is traditionally used as a measure of the “goodness” of a biometric system. It is defined as the point where False Acceptance Rate equals the False Rejection Rate.

FIGS. 6 and 8 establish the inter-individual variability that can be used for identification purposes. i.e., these figures show that the ECGs from different individuals are different in a consistent way and that the feature metrics extracted from these ECGs amplify the characteristics, which can be used for identification. However, they do not establish that for the same subject, the ECG taken at different times under different conditions may not confuse the identification algorithms. In order to verify that this intra-individual variability does not pose a threat to results, a series of long term data files are analyzed. (See the section on data sampling described above for details.) Similar to the general analysis, multiple sections of about 10 cycles were chosen and the metrics compared between the same and different persons. However, for robustness studies, the interval between the chosen samples was increased by a factor of about 100 (compared to our general analysis).

FIGS. 7a and 7b show the results of the robustness studies. The statistics available for long term data are limited. However, FIG. 6 shows remarkable consistency with FIGS. 6 and 8.

TABLE 1
Summary of Results

| | Short | Data | Long | Data |
|---|---|---|---|---|
| | Q_{d} | X² | Q_{d} | X² |
| “Cut” | 9.5 | 300 | 7.5 | 125 |
| Efficiency | 73% | 77% | 79% | 75% |
| FAR | 23% | 24% | 19% | 24% |
| FRR | 27% | 23% | 21% | 25% |
| EER | 25% | 23.5% | 20% | 24.5% |

The results obtained from the studies are tabulated in Table 1. The entries in this table are derived from FIGS. 6, 7a, 7b and 8 and they summarize both the general analysis and the robustness studies. Particular attention should be paid to the following:

1. The [symbol] ² distance between the metric gives better results. This was expected because proper treatment of the measurement errors must reduce the erroneous estimate of significance in distance measurements.

2. The long term data, though statistically limited, gives numbers similar to the short term data. This proves that even relatively long periods (of almost 24 hours), ECG waveform from a person retains distinctive features that the analysis embodying the present invention is able to extract.

It has been shown in the present study that known variabilities in a user's ECG signal may be normalized out to come up with a robust metric. This metric can be used to identify different users. By looking at the data to which access was obtained, it will be seen that about 77% of the wrong combinations are rejected while keeping about 77% of the right combinations (i.e., an Equal Error Rate of 23%). The data used contained medical pathologies where the ECG data really changed during the measurements (i.e., heart conditions manifesting themselves during the measurement time). It is anticipated that the acceptance rate will go up for normal subjects.

Conversely, whereas the data used in the present experiments contains consecutive data for a number of patients under controlled conditions, it may be useful for the database to include data for normal healthy subjects under varying conditions.

Although only a single embodiment of the invention has been described, many variations are possible within the scope of the invention as will be evident to a skilled reader.

References

The disclosure of the following references is incorporated herein in its entirety by reference:

[1] C. J. Harland, T. D. Clark and R. J. Prance. “Electrical potential probes new directions in the remote sensing of the human body”. Measurement Science and Technology, 13:163169, 2002.

[2] Rudi Hoekema, Gerard J. H. Uijen, and Adriaan van Oosterom. “Geometrical Aspects of the Interindividual Variability of Multilead ECG Recordings”. IEEE Transactions on Biomedical Engineering, 48(5), May 2001.

[3] Lena Biel; Ola Pettersson, Lennart Philipson, and Peter Wide. “ECG Analysis: A New Approach in Human Identification”. IEEE Transactions on Instrumentation and Measurement, 50(3):808812, June 2001.

[4] Robert Plonsey Jaakko Malmivuo. “Bioelectromagnetism—Principles and Applications of Bioelectric and Biomagnetic Fields”. Oxford University Press, 1995.

[5] Larry S. Green, Robert L. Lux, Charles W. Haws and others. “E_ects of age, sex, and body habitus on QRS and ST-T potential maps of 1100 normal subjects”. Circulation, 71(2):244253, February 1985.

[6] Gyorgy Kozmann, Robert L. Lux, Larry S. Green. “Sources of Variability in Normal Body Surface Potential Maps”. Cirulation, 79(5):10771083, May, 1989.

[7] Terrence J. Monague, Eldon R. Smith, Douglas A. Cameron and others. “Isointegral Analysis of Body Surface Maps: Surface Distribution and Temporal Variability in Normal Subjects”. Circulation, 63, No. 5 (5):11661171, May, 1981.

[8] Jari Viik. “Diagnostic Properties of Exercise Electrocardiographic Leads and Variables in the Detection of Coronary Artery Disease”. PhD thesis, Tampere University of Technology, 2000.

[9] W.A.H. Engelse and C. Zeelenberg. “A single scan algorithm for QRS-detection and feature extraction”. Computers in Cardiology, 6:3742,1979.

[10] Goldberger AL, Amaral LAN, Glass L, Hausdor_J M, Ivanov P Ch, Mark R G, Mietus J, Moody G B, Peng C K, Stanley H E. “PhysioBank, PhysioToolkit, and Physionet: Components of a New Research Resource for Complex Physiologic Signals”. Circulation, 101(32):e2156220, 2000.

[11] Pablo Laguna, Roger G. Mark, Ary Goldberger and George B. Moody. “A Database for Evaluation of Algorithms for Measurement of QT and Other.

Waveform Intervals in the ECG”. Computers in Cardiology, 24:673676, 1997.

1. A method for identifying a subject comprising the steps of:
(a) obtaining a digitised recording of an electrocardiogram measurement of the subject to be identified, said digitised recording being a cyclic waveform having a peak amplitude;
(b) normalising the digitised recording for reducing variations due to physiological effects;
(c) processing the normalised recording to determine a feature vector in the frequency domain; and
(d) measuring the distance between the determined feature vector and a predetermined feature vector to identify the subject.

2. A method according to claim 1, wherein the step of normalising comprises normalising the peak amplitude of the digitised recording.

3. A method according to claim 1, wherein the digitised waveform has a DC component, the step of normalising comprising removing the DC component.

4. A method according to claim 1, wherein the step of normalising comprises taking an average of the peak amplitude of a number consecutive cycles to reduce statistical errors.

5. A method according to claim 4, wherein the step of normalising comprises aligning the peaks of the cyclic waveforms prior to taking the average of the peak amplitude.

6. A method according to claim 1, wherein the step of normalising comprises resampling the digitised recording.

7. A method according to claim 6, wherein the step of resampling comprises resampling using FFT resampling techniques.

8. A method according to claim 1, wherein the step of normalising comprises obtaining a quadrature distance for each cycle against a running average of the cycles and rejecting a cycle when the distance is more than a predetermined distance until a predetermined number of cycles has been averaged.

9. A method according to claim 1, wherein the step of processing comprises processing the normalised recording to determine a frequency response.

10. A method according to claim 1, wherein the step of processing comprises obtaining the quadratic distance between the feature vector and a predetermined vector.

11. A method according to claim 1, wherein the step of measuring includes obtaining the quadratic distance between the feature vector and a predetermined vector, applying a correction for standard deviations in both vectors to obtain an error-compensated distance and comparing the error-compensated distance with a predetermined value to identify the subject.

12. A system for identifying a subject using a digitised recording of an electrocardiogram measurement of the subject to be identified, said system being arranged to carry out the method according to claim 1.

13. A system for identifying a subject using a digitised recording of an electrocardiogram measurement of the subject to be identified, said digitised recording being a cyclic waveform having a peak amplitude the system comprising:
(a) a normalising device for normalising the digitised recording;
(b) a processor for processing the normalised recording to determine a feature vector in the frequency domain, the processors being arranged to measure the distance between the determined feature vector and a predetermined feature vector to identify the subject.
